(19) United States
(12) Patent Application Publication
(10) Pub. No.: US 2009/0093552 A1
(43) Pub. Date: Apr. 9, 2009

(54) Addition of lecithin as an antistatic agent for the crystallization processing of organic molecules

(76) Inventor: Ivan Lee, Piscataway, NJ (US)

Correspondence Address: MERCK AND CO., INC, P O BOX 2000, RAHWAY, NJ, 07065-0907, US

(21) Appl. No.: 12/287,429

(22) Filed: Oct. 9, 2008

Related U.S. Application Data
(60) Provisional application No. 60/998,203, filed on Oct. 9, 2007.

(51) Int. Cl. A61K 47/12

(52) U.S. Cl. 514/784

(57) ABSTRACT

The present invention relates to the addition of lecithin during the crystallization of organic molecules in organic solvents as an antistatic agent to reduce or eliminate static buildup, thus reducing the potential for explosion and equipment damage during chemical processing.

### CROSS REFERENCE TO RELATED APPLICATIONS

This application claims the benefit of U.S. Provisional Application No. 60/998,203, filed Oct. 9, 2007, the contents of which are incorporated herein by reference in their entirety.

### FIELD OF THE INVENTION

The present invention relates to the addition of lecithin during the crystallization of organic molecules in organic solvents as an antistatic agent to reduce or eliminate static buildup, thus reducing the potential for explosion and equipment damage during chemical processing.

### BACKGROUND OF THE INVENTION

The buildup of static electricity in slurries comprised of organic molecules, for example, active pharmaceutical ingredients (API), in organic solvents is a major safety concern during chemical processing. Usually, non-polar organic solvents including, but not limited to, heptanes, hexanes, and toluene, have a higher propensity to generate and build up static charge. A non-exhaustive list of systems where static electrical charges may exist includes 1) when solids are mixed with solvent; 2) when there is agitation or fluid flow in the system, as in internal recycle or liquid discharge steps; and 3) when the internal surfaces of the equipment are non-conductive such as Teflon-lined hoses and glass-lined reaction vessels. Static electricity build-up and untoward discharge can lead to damage to the crystallization vessels and other equipment, as well as being a potential explosion hazard if vessel inertion is lost.

Lecithin is a naturally occurring surfactant that is often extracted from food sources including soybeans and eggs. Lecithin is used as an emulsifier in the food industry and as an excipient in the pharmaceutical industry. Lecithin is used in pharmaceutical formulations at up to 20% load with no known toxicity or negative impact on human health and is FDA approved for human consumption. It is generally considered safe and is available in both food and pharmaceutical grades. The composition of lecithin is a mixture of phospholipids, glyceride oils and fatty acids and is defined in U.S. Pat. No. 3,257,331 (issued Jun. 21, 1966).

Lecithin's surfactant and charge carrying abilities are well known. Lecithin has been proposed or applied as an antistatic fabric softener agent [U.S. Pat. No. 3,257,331; U.S. Pat. No. 4,808,320 (issued Feb. 28, 1989)]; as an antistatic agent in the jet grinding formulation of micronized biodegradable particles for inhaled drug-delivery systems [U.S. Pat. No. 5,871,771 (issued Feb. 16, 1999)]; as a surfactant in formulating dye dispersions in aqueous solutions [U.S. Pat. No. 6,045,986 (issued Apr. 4, 2000)]; as well as a charge control agent for color printer toner formulations [U.S. Pat. No. 5,153,090 (issued Oct. 6, 1992); U.S. Pat. No. 5,411,833 (issued May 2, 1995); U.S. Pat. No. 4,762,764 (issued Aug. 9, 1988); U.S. Pat. No. 7,018,769 (issued Mar. 28, 2006); and U.S. Pat. No. 5,693,441 (issued Dec. 2, 1997)].

Lecithin and other organic and inorganic compounds have been identified and used as antistatic agents, primarily in gas-phase organometallic-catalyzed polymerization reactions. [U.S. Pat. No. 5,264,313 (issued Nov. 23, 1993); U.S. Pat. No. 5,034,480 (issued Jul. 23, 1991); U.S. Pat. No. 5,410,002 (issued Apr. 25, 1995); EP 0811638A3 (Dec. 10, 1997); and U.S. Pat. No. 4,012,574 (issued Mar. 15, 1977)]. Though other polymeric organic additives such as mixtures of polysulfones and polyamines are commonly used to increase the conductivities of various hydrocarbon fuels during transportation and handling [U.S. Pat. No. 3,917,466 (issued November 1975); U.S. Pat. No. 4,029,480 (issued June 1977)], they have not been screened or approved for human consumption or pharmaceutical applications.

Lecithin has not been used as an antistatic agent in the crystallization of organic molecules in organic solvents. It has been used as a crystal-growth modifier in the crystallization of alkali metal bicarbonate salts in aqueous solutions [U.S. Pat. No. 6,042,622 (issued Mar. 28, 2000)]; and as an additive in the formation of lipid nanoparticles in drug-delivery systems [M. A. Schubert, et al., Int. J. Pharma., 298 (1): 242-254 (2005)].

The advantage of using lecithin as an antistatic agent in the present invention, compared to other commercially available antistatic agents, is that lecithin is already commonly used as an excipient in pharmaceutical formulations, so there is no safety or regulatory concern related to its inclusion at the load levels indicated in the present invention. The load levels of the present invention are from about 0.005% to about 0.05% by weight. The load levels of the present invention are an order of magnitude lower than the load levels used in the jet grinding formulation of micronized biodegradable particles for inhaled drug-delivery systems (0.5% by weight). The present load levels are also considerably lower than the concentrations used in the formation of solid-lipid nanoparticles in drug-delivery systems, which is at least 10% by weight.

Previously, one solution to static build up in non-polar API slurries was to use metal reactor vessels. Generally, very expensive metal alloys, such as HASTELLOY C, must be used as the material of new vessel construction because of the acidity of some API slurries. The use of specialized metal alloys, such as HASTELLOY C, in vessel construction is both time-consuming and expensive. Using lecithin, as in the present invention, allows the use of general purpose glass-lined vessels in the API process, incurring no additional cost or time penalties for new construction.

Another previous possible solution to static buildup in API slurries was to change solvent systems. For many processes there is no suitable alternative solvent solution which is conductive enough and still meets the specific needs of the process. Using lecithin allows one to keep the original solvents, optimized for the specific process, without adversely affecting such properties as the API's solubility, purity, and crystal growth.

### SUMMARY OF THE INVENTION

The present invention relates to the addition of lecithin, a food grade antistatic agent, to slurries of non-polar solvents and organic compounds for processing, such as in the crystallization of active pharmaceutical ingredients.

Static buildup during the crystallization of organic compounds in organic solvents presents a safety risk of fire or explosion, as well as the potential for costly glass damage. Furthermore, additional capitol expenditure and construction time may be needed to construct alternative vessels, such as those containing HASTELLOY C, since stainless steel may not meet all the processing requirements, especially when acidic solutions are employed.

The propensity of static buildup is directly related to the conductivity of the solvent system, and is especially high for non-polar organic solvents. Here, lecithin is used as an antistatic agent due to its relatively high solubility in non-polar organic solvents, as well as its charge carrying capabilities when dissolved in said solvents.

Lecithin has the advantage over other commercial antistatic agents due to its abundant history of routine use in the food and pharmaceutical industry, even in formulations, thus reducing the regulatory obstacles associated with additives introduced into the processing of active pharmaceutical ingredients. There are several food and pharmaceutical grades of lecithin suitable for the present invention. Lecithin has been used in pharmaceutical formulations at up to 20% load with no known toxicity or negative impact on human health.

### BRIEF DESCRIPTION OF THE DRAWINGS

FIG. 1: Plot of lecithin concentration versus conductivity in neat solvents showing linear relationship.

FIG. 2: Plot of lecithin concentration versus conductivity for mixtures containing organic Compound A in 1:1 toluene:heptane and organic Compound B in 1:2 toluene:heptane showing a linear relationship similar to that of neat solvents for low surface area solids (<1 sqm/g).

FIG. 3: Plot of lecithin concentration versus conductivity for neat heptane, and slurries containing organic Compound A, B or C.

FIG. 4: Plot of conductivity versus lecithin load/solids area for the organic Compound C.

FIG. 5: Crystals of organic Compound A grown both with and without lecithin. Crystals of organic Compound A exhibit a preferred particle size distribution upon addition of lecithin.

FIG. 6: Crystals of organic Compound B grown both with and without lecithin. Crystals of organic Compound B exhibit similar particle size distributions regardless of presence of lecithin.

FIG. 7: Crystals of organic Compound C grown both with and without lecithin. Crystals of organic Compound C exhibit similar particle size distributions regardless of presence of lecithin.

### DETAILED DESCRIPTION OF THE INVENTION

The present invention relates to the addition of lecithin, a food grade antistatic agent, to slurries of non-polar solvents and organic compounds for processing, such as in the crystallization of active pharmaceutical ingredients. The present invention addresses static buildup during the crystallization of organic compounds in organic solvents, which presents a safety risk of fire or explosion, as well as the potential for additional capitol expenditure and construction time when alternative vessels are necessary.

Lecithin has the advantage over other commercial antistatic agents due to its routine use in the pharmaceutical industry, thereby reducing the regulatory obstacles associated with additives introduced into the processing of active pharmaceutical ingredients. There are several food or pharmaceutical grades of lecithin suitable for the present invention.

Lecithin is a naturally occurring compound found in various food sources such as eggs and soybeans. Lecithin has been used in pharmaceutical formulations at up to 20% load with no known toxicity or negative impact on human health. Lecithin is FDA approved for use as an excipient and is used as an emulsifier and surfactant in the food industry.

The composition of lecithin is a mixture of organic compounds such as glycolipids, triglycerides, and phospholipids, such as phosphatidylinositol, alpha-phosophotidylcholine, and phosphatidylethanolamine, and is defined in U.S. Pat. No. 3,257,331 (issued Jun. 21, 1966). In one embodiment of the present invention, alpha-phosphatidylcholine is used as the basis for determining lecithin load. In this embodiment, the lecithin used is derived from soybeans and contains more than about 30% alpha-phosphatidylcholine, as determined by TLC. More expensive lecithin grades, which contained higher concentrations of alpha-phosphatidylcholine, did not enhance the present invention.

In one embodiment of the present invention, organic Compound A is a free base with a molecular weight of about 359 g/mol. Organic Compound A is relatively non-polar and is soluble in toluene at about 100 g/L. Organic Compound A crystallizes in several different morphologies, and it is the plate-like form that is used in production. Organic Compound A has a surface-area of 1 sqm/g.

In one embodiment of the present invention, organic Compound B is a free base converted from the original HCl salt and has a molecular weight of about 493 g/mol. Organic Compound B is slightly polar and is soluble in toluene at about 180 g/L. Organic Compound B crystallizes in long rods and has a surface-area of 0.47 sqm/g.

In one embodiment of the present invention, organic Compound C is a free acid and has a molecular weight of about 527 g/mol. Organic Compound C is slightly polar and is soluble in toluene at more than 100 g/L. The slurry containing organic Compound C is conductive when dissolved in a toluene/heptane mixture due to liberated water molecules present in the crystal lattice. Organic Compound C crystallizes in long rods and has a surface-area of about 10 sqm/g.

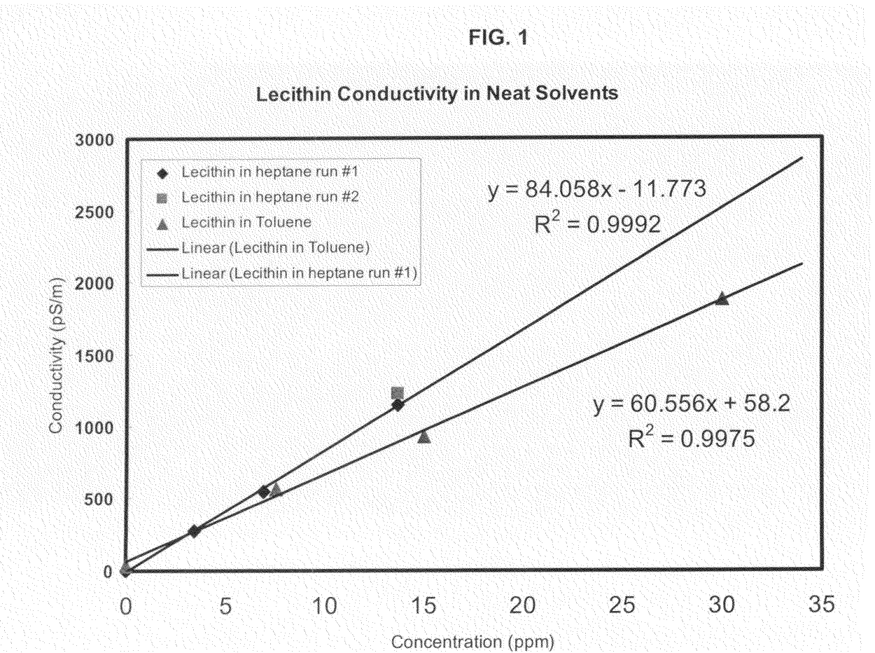

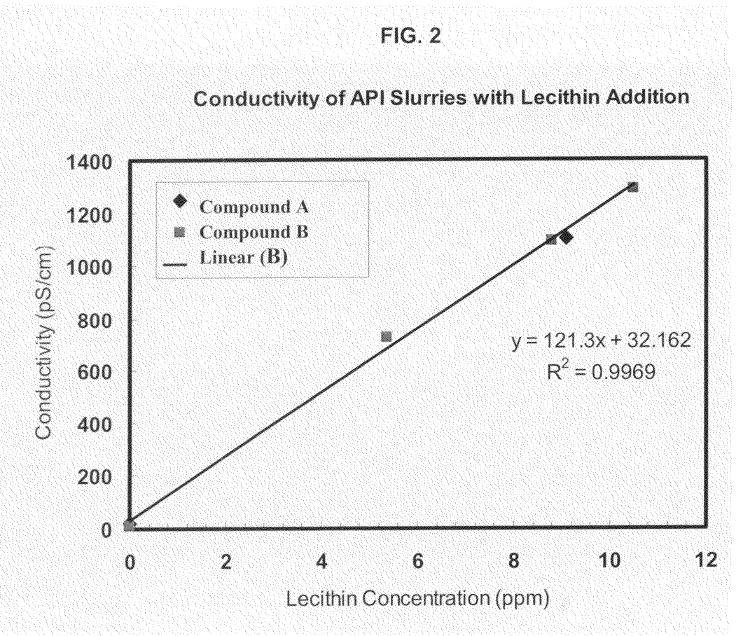

In one embodiment of the present invention, results show that addition of lecithin at part per million (ppm) levels is sufficient to increase the solution and the API slurry conductivity to above the target conductivity threshold of 1000 pS/m. In a further embodiment, the relationship between the conductivity of neat solvents and lecithin concentration is linear. See FIG. 1. In another embodiment of the present invention, smaller surface area solids (<0.5-1 sqm/g), including compounds such as organic Compound A and organic Compound B, also possess a linear relationship between conductivity and lecithin concentration. See FIG. 2.

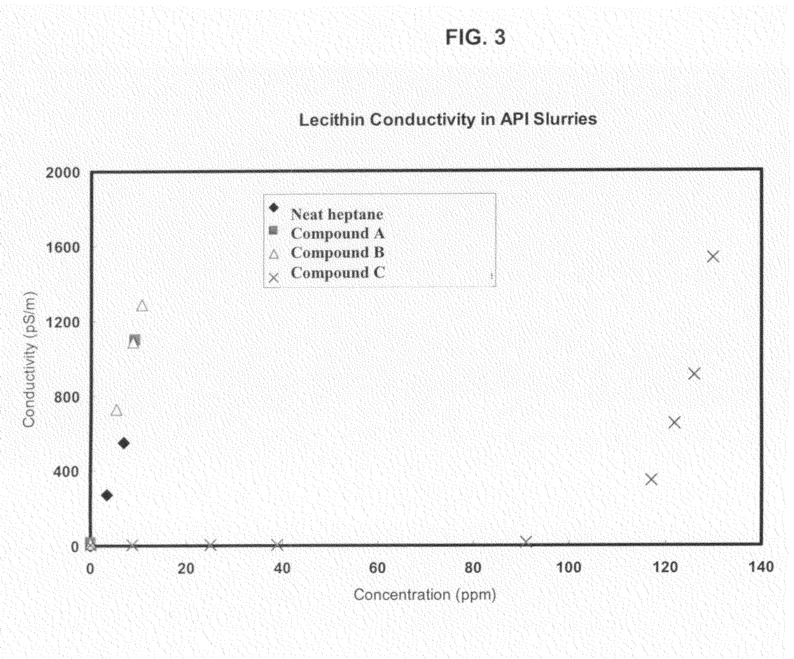

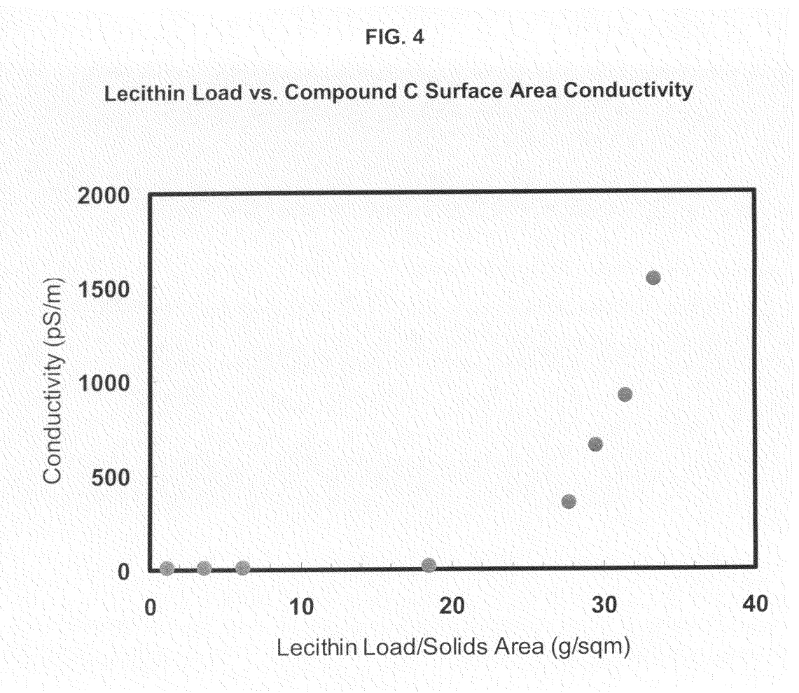

In one embodiment of the present invention, the amount of lecithin required to reach the conductivity threshold is related to the surface area of the API solids. Sufficient lecithin must be added to coat the solid surface with monolayer coverage before the excess lecithin is solvated and electrically active in solution. See FIG. 3. Once lecithin has been added in sufficient quantity to become solvated in the liquid phase and capable of carrying electric charge, the linear relationship between slurry conductivity and lecithin load is restored. See FIG. 4.

The conductivity of the slurries was measured by a Scientifica Model 600 conductivity meter; with the defined threshold for conductivity at >1000 pS/m. In one embodiment of the present invention, lecithin loads up to 400 ppm may be required for API solids with a large surface area (>2-3 sqm/g), but the preferred range is from about 10 to about 50 ppm. In this embodiment of the present invention, lecithin is comparable to other antistatic agents reaching a conductivity threshold from about 10 ppm to about 30 ppm.

In further embodiments of the present invention, comparison crystallization studies were conducted both with and without lecithin for several diverse organic, API, compounds in non-polar solvents. The addition of lecithin did not negatively impact the crystallization process or the desired physical properties of the resultant API solids tested.

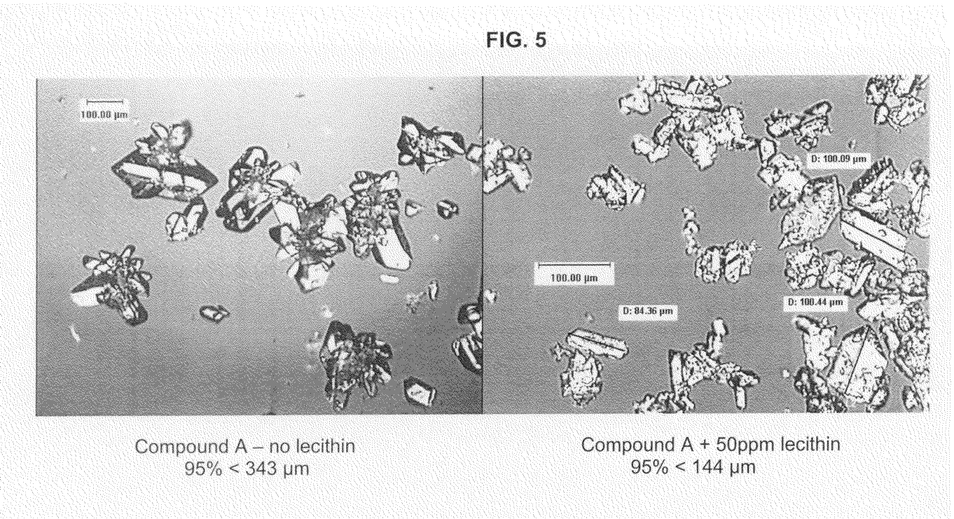

In one embodiment of the present invention, Compound A was crystallized both with and without the addition of lecithin in 1:1 toluene:heptane. Crystals of Compound A exhibit a preferred particle size distribution upon addition of lecithin as compared to crystals grown in the absence of lecithin. In FIG. 5, crystals of Compound A grown without lecithin resulted in 95% of the crystals having a particle size of less than 343 μm. However, the crystals of Compound A grown in the presence of 50 ppm lecithin had a comparable size distribution to that of Compound B (with and without lecithin) with 95% of the crystals having a particle size of less than 144 μm.

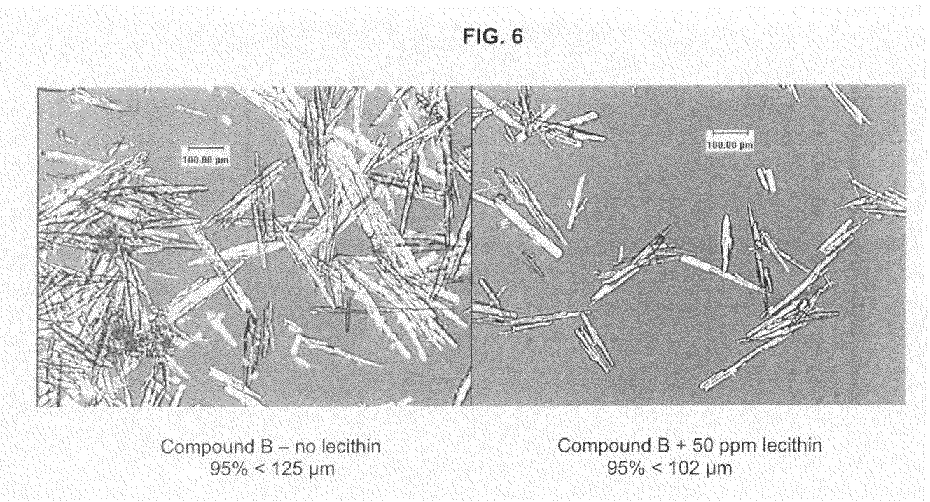

In another embodiment of the present invention, Compound B was crystallized both with and without the addition of lecithin in 1:2 toluene:heptane. Crystals of Compound B exhibit a similar particle size distribution regardless of the presence of lecithin. In FIG. 6, crystals of Compound B grown without lecithin resulted in 95% of the crystals having a particle size of less than 125 μm. The crystals of Compound B grown in the presence of 50 ppm lecithin had a comparable size distribution with 95% of the crystals having a particle size of less than 102 μm.

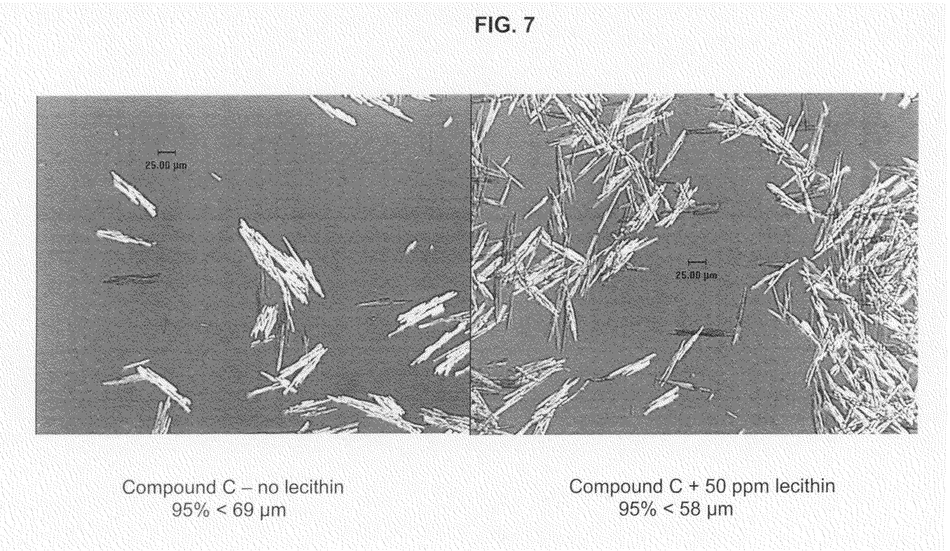

In another embodiment of the present invention, Compound C was crystallized both with and without the addition of lecithin in heptane. Crystals of Compound C exhibit a similar particle size distribution regardless of the presence of lecithin. In FIG. 7, crystals of Compound C grown without lecithin resulted in 95% of the crystals having a particle size of less than 69 μm. The crystals of Compound C grown in the presence of 50 ppm lecithin had a comparable size distribution with 95% of the crystals having a particle size of less than 58 μm.

What is claimed is:

1. A process comprising
a) forming a slurry consisting of dissolving at least one organic compound in an organic solvent; and
b) adding lecithin as an antistatic agent to the said slurry in sufficient quantity to reach a target conductivity threshold of about 1000 pS/m.

2. A process according to claim 1 where the organic compounds are active pharmaceutical ingredients.

3. A process according to claim 2 where the organic solvents are non-polar solvents including, but not limited to, heptanes, hexanes, and toluene and mixtures thereof.

4. A process according to claim 3 where the amount of lecithin added is from about 400 ppm to about 5 ppm.

5. A process according to claim 4 where the amount of lecithin added is from about 100 ppm to about 10 ppm.

6. A process according to claim 5 where the amount of lecithin added is from about 50 ppm to about 10 ppm.
